(19) United States
(12) Patent Application Publication
(10) Pub. No.: US 2026/0157681 A1
(43) Pub. Date: Jun. 11, 2026

(54) MUSCLE-PERFORMANCE MONITORING SYSTEM

(71) Applicant: AthleteIQ, Inc., Dover, DE (US)

(72) Inventors: Leonardo Garofalo, Campobasso (IT); Sophie Defauw, Puerto Aventuras (MX); Benjamin David White, Oxford (GB)

(73) Assignee: AthleteIQ, Inc., Dover, DE (US)

(21) Appl. No.: 18/975,861

(22) Filed: Dec. 10, 2024

(51) Int. Cl. A61B 5/397; A61B 5/00; A61B 5/259; A61B 5/296

(52) U.S. Cl. CPC A61B 5/397; A61B 5/259; A61B 5/296; A61B 5/7257; A61B 5/7264; A61B 5/7435; A61B 5/7475; A61B 2562/0217; A61B 2562/164; A61B 2562/166

(57) ABSTRACT

A device for accessing a muscle-fatigue status of a muscle is provided. The device has a removably-adhesive layer that surrounds a pair of bi-polar electrodes. The device further includes a processing unit that analyzes a surface electromyography signal detected by the bi-polar electrodes and determines a muscle-fatigue status of a muscle of the user.

### BACKGROUND

Monitoring muscle fatigue and muscle performance during a workout is critical to achieving good results and avoiding injury. A good athletic trainer often has a natural ability to assess how far to push someone the athletic trainer is guiding through a workout. The trainer must balance pushing the athlete enough to stimulate muscle adaptation whilst, at the same time, carefully avoiding excessive strain that could lead to injuries. What is needed is a device that allows athletes and other gym-goers to monitor their own muscle performance and muscle fatigue during a workout. Such a device would ideally be configured to help the gym-goer avoid overtraining and undertraining and to allow optimal personalized training.

### SUMMARY

Disclosed herein are embodiments of a muscle-performance monitoring system. In certain arrangements, the muscle-monitoring system can include a wearable device with a removably-adhesive layer that surrounds a plurality of electrodes configured to detect a surface electromyographic signal from a tissue of the user that underlies the wearable device.

Also disclosed are methods of operating a muscle-monitoring system to monitor the muscle performance of a user of the muscle-monitoring system. In certain arrangements, the system processes a detected sEMG biophysical signal of the user by extracting a window from the signal and performing a short-time Fourier transform on this window. The resulting spectrogram is then analyzed by a neural network. The neural network estimates the user's level of muscle fatigue, and this information is used to check whether the user is at risk of injury and to give personalized workout recommendations.

### BRIEF DESCRIPTION OF THE DRAWINGS

Embodiments of the present disclosure will now be described hereinafter, by way of example only, with reference to the accompanying drawings in which:

FIG. 1 illustrates a sensor device of a muscle-monitoring system in use to monitor a muscle fatigue of the system user, according to some aspects of the present disclosure.

FIG. 2 illustrates a sensor device of a muscle-monitoring system stored on a wristband of the user when the system is not in use to monitor a muscle fatigue of the user, according to some aspects of the present disclosure.

FIG. 3 illustrates a muscle-monitoring system for monitoring a muscle fatigue of a user of the system, according to some aspects of the present disclosure.

FIG. 4A illustrates a top view of a wearable device of a muscle-monitoring system, according to some aspects of the present disclosure.

FIG. 4B illustrates a side view of the wearable device of FIG. 4A, according to some aspects of the present disclosure.

FIG. 4C illustrates an electrode-face view of the wearable device of FIG. 4A, according to some aspects of the present disclosure.

FIG. 5A illustrates a top view of a storage clip for a wearable device of a muscle-monitoring system, according to some aspects of the present disclosure.

FIG. 5B illustrates an end view of the storage clip of FIG. 5A, according to some aspects of the present disclosure.

FIG. 5C illustrates a bottom view of the storage clip of FIG. 5A, according to some aspects of the present disclosure.

FIG. 6A illustrates a side view of an adhesive layer of a wearable device of a muscle-monitoring system, according to some aspects of the present disclosure.

FIG. 6B illustrates a top view of an adhesive layer of a wearable device of a muscle-monitoring system, according to some aspects of the present disclosure.

FIG. 6C illustrates a side view of an adhesive layer of a wearable device of a muscle-monitoring system, according to some aspects of the present disclosure.

FIG. 6D illustrates a top view of an adhesive layer of a wearable device of a muscle-monitoring system, according to some aspects of the present disclosure.

FIG. 6E illustrates a top view of an adhesive layer of a wearable device of a muscle-monitoring system, according to some aspects of the present disclosure.

FIG. 7A illustrates a wearable device of a muscle-monitoring system attached to a skin of a user to monitor a muscle-fatigue level of an underlying muscle, according to some aspects of the present disclosure.

FIG. 7B illustrates schematically a signal-processing method of a muscle-monitoring system, according to some aspects of the present disclosure.

FIG. 7C illustrates schematically a neural-network processing method of a muscle-monitoring system, according to some aspects of the present disclosure.

FIG. 8 illustrates schematically a neural-network-processing architecture of a muscle-monitoring system, according to some aspects of the present disclosure.

### DETAILED DESCRIPTION

### Overview

This disclosure relates generally to a system configured to monitor muscle performance (e.g., fatigue status) of one or more muscles during a workout. In some aspects, the system of the present disclosure can include a wearable device that adheres removably to the wearer's skin, over a muscle of interest, allowing the wearer of the device to re-locate the patch throughout a training session (e.g., a workout comprising different exercises) and thereby change which muscle the wearable device monitors. In some arrangements, the system can include a plurality of wearable devices, allowing a plurality of muscles to be monitored simultaneously.

FIG. 1 depicts a non-limiting, illustrative embodiment of a muscle-monitoring system 100, according to some aspects of the present disclosure. The system 100 can include a wearable device 200 that can be configured to adhere reversibly to the skin 2 of the wearer, as described herein. FIG. 1 shows the wearable device 200 can be positioned on the skin 2 of the arm 4 of a wearer for use in monitoring the performance of the underlying bicep muscle. The dashed box in FIG. 1 indicates schematically that the wearable device 200 can be configured to adhere to the wearer's skin 2 through a removably-adhesive layer 210. The removably-adhesive layer 210 can be configured to reversibly secure the wearable device 200 over the monitored muscle 6, as described herein.

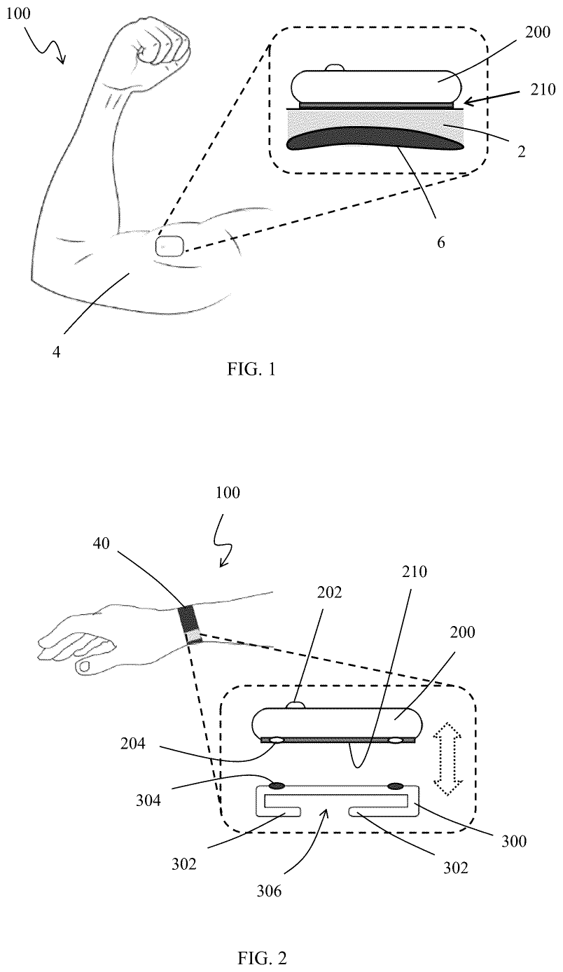

FIG. 2 depicts a non-limiting, illustrative embodiment of a muscle-monitoring system 100, according to some aspects of the present disclosure. FIG. 2 illustrates that the wearable device 200 can be configured for storage on a user-worn strap 40 when the user is not using the system 100 to monitor performance of a muscle. In some aspects, the user can removably secure the wearable device 200 to a storage clip 300 by engaging a device anchor 204 of the wearable device 200 with a corresponding strap anchor 304 of the storage clip 300. With reference to the insert of FIG. 2, the storage clip 300 can have a pair of opposing wings 302 that extend incompletely toward one another, forming a gap 306 through which the lateral edges of the strap 40 can be tucked to attach the storage clip 300 onto the strap 40. The device anchors 204 can be aligned with, and coupled reversibly to, the clip anchors 304 to secure the wearable device 200 onto the clip anchor 304. For example, in some arrangements the device anchor 204 can be configured as a protruding snap button that fits within a collar-like receiving button of corresponding clip anchor 304. In some arrangements, the device anchor 204 and the corresponding clip anchor 304 can include interacting magnets of opposite polarity. As shown in FIG. 2, the adhesive layer 210 can be disposed between the device 200 and the storage clip 300 when the wearable device 200 is stored on the storage clip 300.

Electromyography (EMG) is a diagnostic and research tool used to record and analyze the electrical activity of skeletal muscle. Surface electromyography (sEMG) uses sensors placed on the skin to non-invasively monitor the electrical activity produced by the underlying skeletal muscle. In some aspects, the system 100 of the present disclosure can evaluate muscle activity by using sEMG to measure electrical impulses transmitted to the muscle fibers via motor neurons. In some arrangements, the system 100 can use a custom signal-processing and machine-learning pipeline to quantify muscle fatigue during an aerobic exercise or during an anaerobic exercise, as described herein. Continuous monitoring of local muscle fatigue during performance of work (e.g., exercise) can be achieved by measuring myoelectric activity of particular muscles by sEMG. Muscle fatigue leads to a decrease of fiber conduction velocity, increased activity of slow motor units as fast ones tire, and motor units synchronization. These changes affect the sEMG signal. In some aspects, the system 100 is configured to determine muscle fatigue based on neural-network and machine-learning processing of the sEMG signal detected by the wearable device 200 during exercise of the underlying muscle. Processing methods suitable for sEMG-based muscle fatigue evaluation can include time domain methods (e.g., Mean Absolute Value, Root-Mean-Square for analyzing sEMG amplitude), and frequency domain methods (e.g., Fourier-based spectral estimators, parametric-based spectral estimators), as described herein.

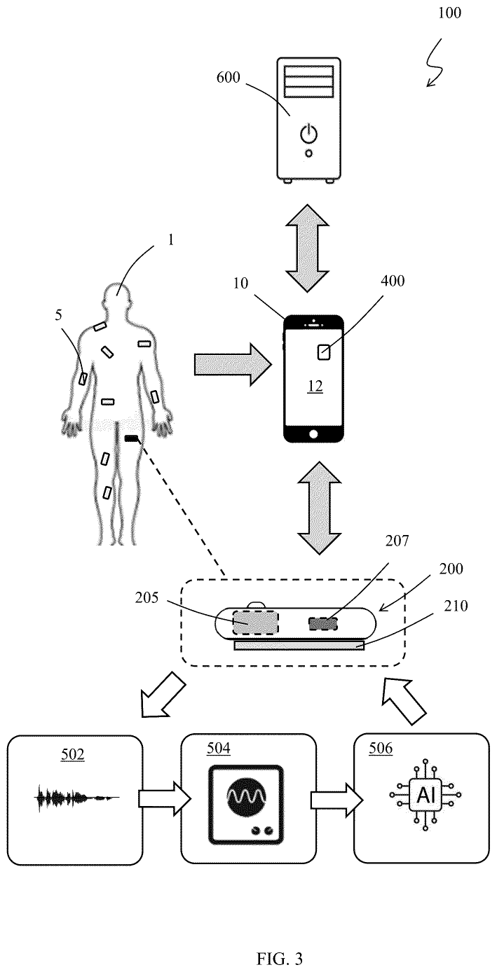

FIG. 3 depicts a non-limiting, illustrative embodiment of a muscle-monitoring system 100, according to some aspects of the present disclosure. As described herein, the system 100 can include an application program 400 (also referred to herein as an “app”) that is configured to run on a computing device 10 (e.g., mobile phone) and allow the user 1 to interact with the system 100, for example through a graphical user interface (GUI) displayed on a display screen 12 of the computing device 10. In FIG. 3, the user 1 is shown with a wearable device 200 adhered to the user's skin over a muscle of the user's upper leg. As indicated by the open boxes in FIG. 3, the user 1 can position the wearable device 200 at other skin locations 5 to monitor a muscle under that skin location 5. As described herein, the wearable device 200 can include a removably-adhesive layer 210 configured to allow the user 1 to move the wearable device 200 to another location (e.g., an open box in FIG. 3) during a workout.

FIG. 3 further illustrates that in use the wearable device 200 can include one or more processors, which for the sake of simplicity are referred to herein as a “processor unit 205”. The processor unit 205 can include one or more processors to perform the methods described herein such as, for example, a signal-processing processor can be one or more processors collectively operating to be configured to detect, through the skin of the user 1, a biophysiological signal 502 (e.g., myoelectric voltage) associated with an activity of the underlying muscle. The wearable device 200 can include signal-processing software 504, as described herein, configured to process the detected biophysiological signal 502. In some arrangements, the signal-detection and signal-processing software processes can be encoded within the processing unit 205 disposed within the wearable device 200, as indicated in FIG. 3.

As indicated in FIG. 3 and described herein, the processor unit 205 of the wearable device 200 can include a specialized artificial-intelligence (AI) chip that allows the wearable device 200 to run neural networks locally (e.g., using the processor unit 205 of the device 200). The wearable device 200 can include a communications module 207 configured to communicate (e.g., wirelessly exchange data) with the mobile device 10 or with a remote server 600. In some arrangements, the device 200 does not have a built-in cellular capability such as the ability of the device 200 to connect to the internet by itself, but rather the device 200 communicates with a mobile device (e.g., mobile phone), which then allows the device 200 to exchange data with the remote server 600. The system 100 can include an artificial-intelligence (AI) module 506 that uses artificial intelligence (e.g., neural networks, machine learning) to analyze the biophysiological signal 502. In some arrangements, the AI-module 506 can analyze the biophysiological signal 502 after the detected biophysiological signal 502 has been processed with the signal processing software 504, as described herein.

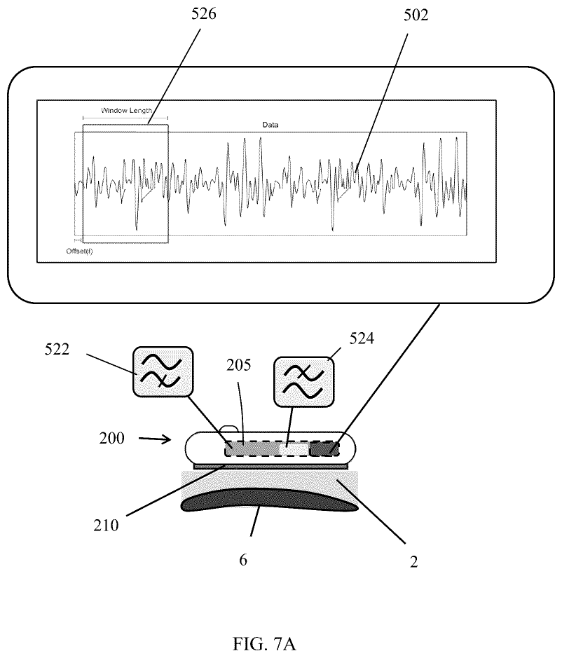

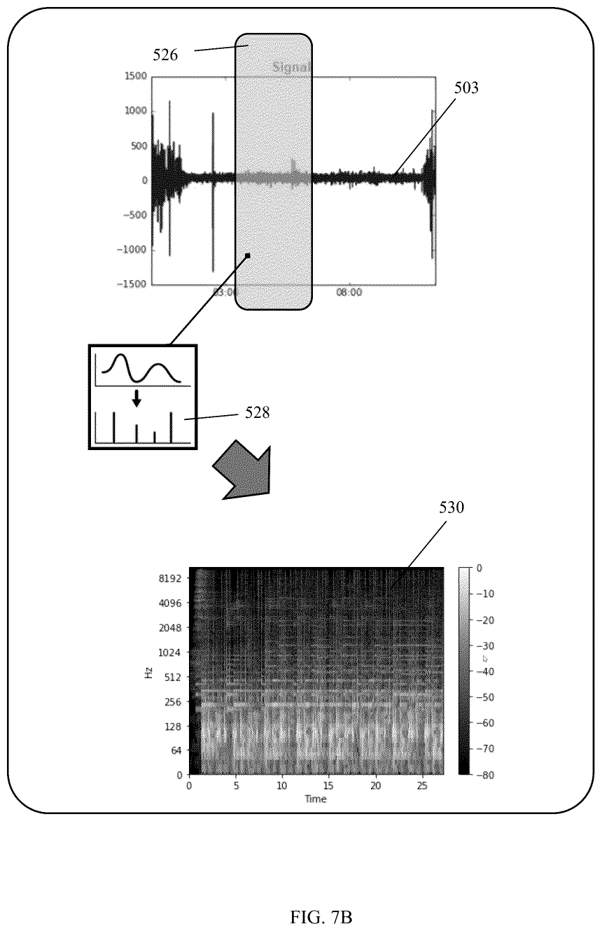

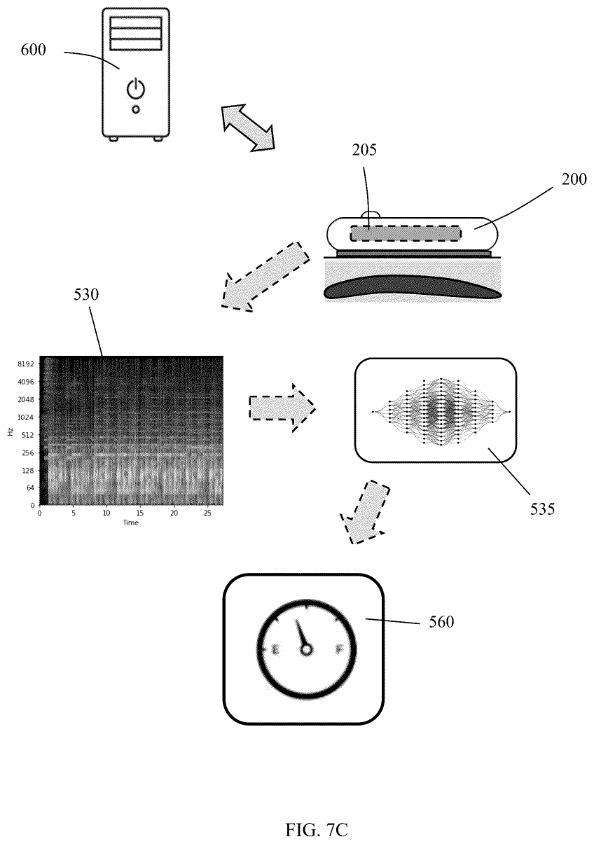

The filled arrows in FIG. 3 illustrate that the system 100 can allow a user 1 to interact with a mobile device 10 to monitor muscle fatigue of various muscles (e.g., as indicated by open-box locations) during a workout. The wearable device 200 can measure a biophysical signal 502 (e.g., a sEMG signal) and process the signal 502 for real-time assessment of muscle fatigue using a signal-processing software 504 and an AI-module 506 and then check for potentially injurious conditions arising from excessive muscle fatigue. If the real-time assessment of muscle fatigue determines a dangerous condition exists due to muscle fatigue (e.g., the current fatigue levels deviate significantly from the user's typical fatigue patterns for the muscle examined), the wearable device 200 can communicate with the mobile device 10 (e.g., through the communications module 207) to provide the user 1 a real-time alert of the dangerous condition. In some arrangements, real-time monitoring and alert notifications can be performed using the remote server 600. The system 100 can adapt to the user 1 and improve its accuracy by fine-tuning the weights of the neural network based on the user's exercise data acquired over time. In some arrangements, the device 200 can run locally the muscle fatigue algorithm (e.g., FIG. 7C) in real time. This data can be then sent to the remote server 600, which leverages both data collected from the user's past workouts and real-time muscle fatigue data from the current workout session to generate personalized, real-time workout recommendations such as adjustments to the intensity of the workout, exercise substitution, recovery time suggestions, progressive overload management recommendations, and similar recommendations. This recommendation can be done on the remote server 600 through traditional software together with machine learning algorithms.

In some aspects, the system 100 can be configured to provide the user 1 with personalized workout recommendations or information, such as, for example: a dynamic intensity modulation recommendation where, based on the biophysical signals detected by the device 200, the system 100 can suggest real-time adjustments to an intensity of the workout (e.g., reduce weight load, increase rest intervals) to reduce the risk of injury; an exercise substitution recommendation where if a muscle group is fatigued, the system 100 can recommend alternative exercises that target the muscles in a less strenuous way for the user 1; a recovery time suggestion in which by analyzing the fatigue data, the system 100 can recommend optimal rest periods between sets and sessions to ensure adequate muscle recovery; a progressive overload management recommendation in which the system 100 can ensure the user's training follows a progressive overload principle without overtraining by adjusting intensity based on recovery and muscle fatigue metrics.

### Hardware

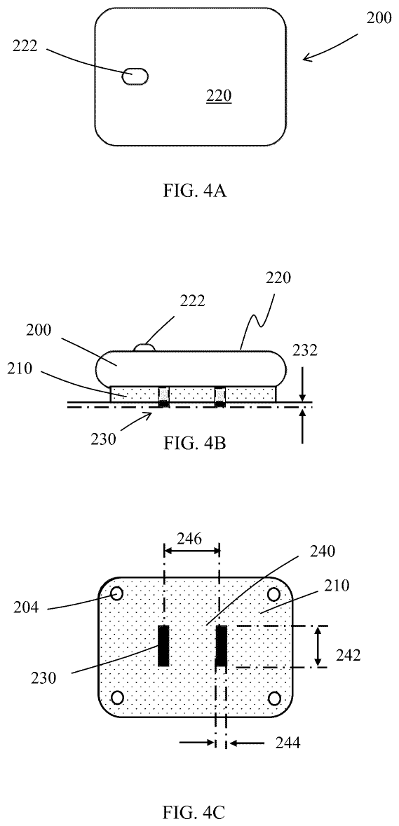

FIGS. 4A-4C depict different views of an illustrative, non-limiting example of a wearable device 200 of the system 100, according to some aspects of the present disclosure. FIG. 4A illustrates a top view of wearable device 200, with a user-viewable surface 220 of the device 100 visible, as would be the case when the user is using the device 200 to monitor muscle fatigue. The wearable device 200 can include a power button 222 configured to allow a user to toggle the device 200 between an on mode and an off mode of the device 200. FIG. 4B illustrates that the wearable device 200 can include one or more electrodes 230 that are surrounded by the removably-adhesive layer 210. In the illustrated embodiment, the wearable device 200 has two electrodes 230, each of which extends a protrusion distance 232 beyond the in-use skin-facing side of the adhesive layer 210. In some aspects, the device 200 can be characterized by a protrusion ratio that is defined as the protrusion distance 232 relative to the thickness of the removably adhesive layer 210. In the illustrated arrangement of FIG. 4B, the protrusion ratio would be about 0.2 because the electrode 230 protrudes such that about one-fifth of the electrode 230 is uncovered by the adhesive layer 210 when viewed from the side. In some arrangements, the wearable device 200 can have a protrusion ratio having a value of: 0.05, 0.1, 0.2, 0.3, and values between any of these values. In some arrangements, the removably-adhesive layer 210 can be configured to completely cover the skin-facing surface of the device 200 except for the places in the adhesive layer 210 through which the electrodes 230 protrude.

In the illustrated arrangement of FIG. 4C, the removably-adhesive layer 210 surrounds a pair of electrodes 230 (e.g., bi-polar electrodes) that protrude beyond the in-use skin-facing surface of the removably-adhesive layer 210. In the illustrated arrangement, the electrodes 230 can be characterized by a span dimension 242, a thickness dimension 244, and a separation dimension 246. In some arrangements, the wearable device 200 can be flexible and configured to bend and follow the movement of the user's skin while maintaining contact of the electrodes 230 and removably-adhesive layer 210 with the skin of the user as the user moves and performs exercises. For example, the wearable device 200 can be made of flexible materials such as, for example, a flexible printed circuit board and a flexible housing. The flexibility of the device 200 can be achieved by using materials that have a low modulus of elasticity. Additionally and alternatively, the flexibility of the wearable device 200 can be achieved by reducing the thickness of the materials so that the materials offer reduced bending resistance to the motion of the user.

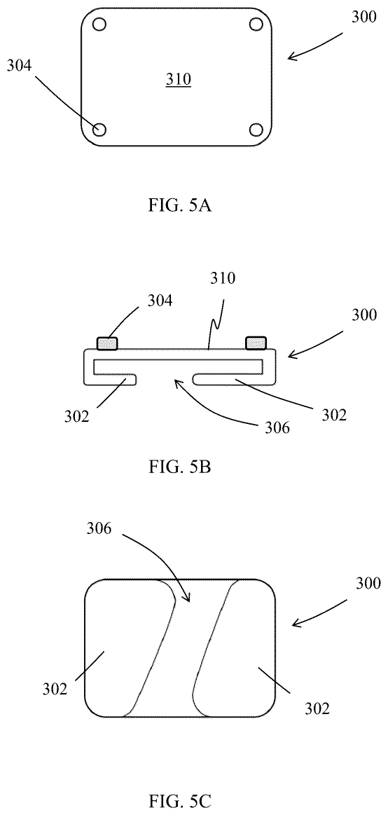

FIGS. 5A-5C depict different views of an illustrative, non-limiting example of a storage clip 300 for securing the wearable device 200 shown in FIG. 2, according to some aspects of the present disclosure. FIG. 5A illustrates the storage clip 300 can have a device-facing surface 310 configured with anchors 304 for securing the device 200 to the storage clip 300, as described herein. FIG. 5B illustrates the storage clip 300 can have a gap 306 formed between two opposing wings 302. The gap 306 can be sized to allow a band (FIG. 2) to be inserted into the open area underneath the opposing wings 302, as described herein. FIG. 5C illustrates that the opposing wings 302 can be sized to retain a band inserted under the opposing wings 302 through the gap 306, as described herein.

### Adhesive

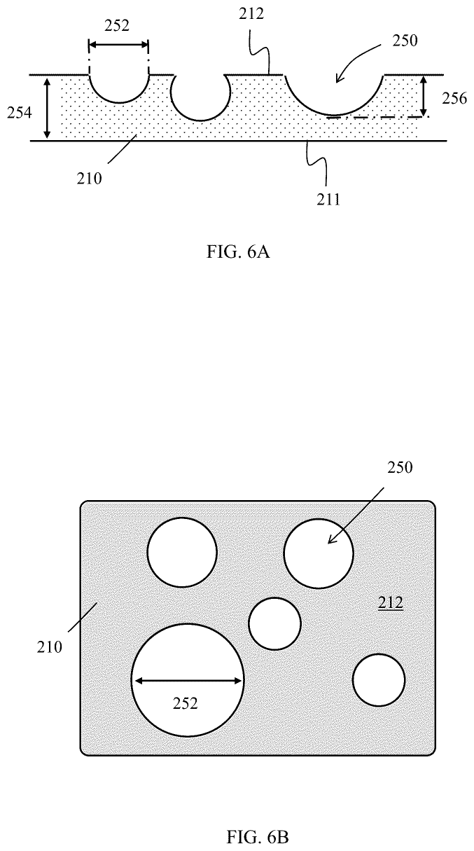

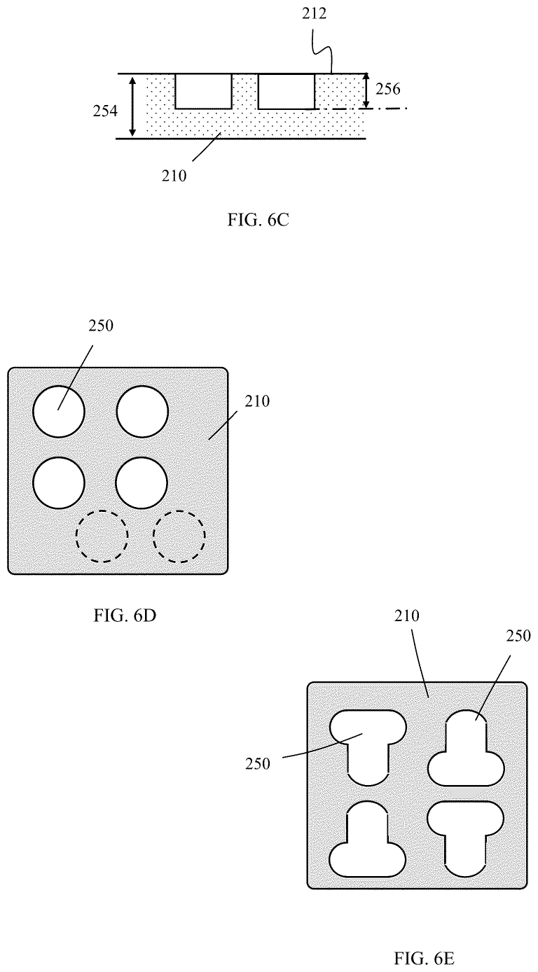

FIG. 6A depicts a side cross-sectional view of an illustrative, non-limiting example of a removably-adhesive layer 210, according to some aspects of the present disclosure. In some aspects, the removably-adhesive layer 210 can comprise a plurality of voids 250 that open to the skin-facing surface 212 of the removably-adhesive layer 210. In some aspects, the voids 250 can be semi-spherical in shape and can be manufactured by molding the removably-adhesive layer 210 onto a surface having sintered microbeads or by introducing spherical beads onto the skin-facing surface 212 during curing of the removably-adhesive layer 210, with subsequent removal of the introduced beads afterwards. FIG. 6A illustrates that the voids 250 can be characterized by a void diameter dimension 252 at the skin-facing surface 212. However, the void diameter dimension 252 need not be the true diameter of the void 250 (as shown in the left-most void 250 of FIG. 6A), but instead the true diameter of the void 250 can be inferior to the skin-facing surface 212 (as shown in the center void 250 of FIG. 6A) or can be superior to the skin-facing surface 212 (as shown in the right-most void 250 of FIG. 6A).

In some aspects, the adhesion of the removably-adhesive layer 210 can be based on a hydrogel network containing glycerol that physically associates with OH groups on the surface of the skin. The removably-adhesive layer 210 can be made of a robust material that is stretchy (e.g., high elastic modulus) and exhibits shape memory properties providing the removably-adhesive layer 210 a reusability aspect. In some aspects, the removably-adhesive layer 210 can be designed to work well with EMG devices for muscle monitoring and other body monitoring purposes. The size and shape of the removably-adhesive layer 210 can be easily shaped, molded, and micropatterned, as described herein. In some aspects, the removably-adhesive layer 210 can be micropatterned with suction-cup-structures to enhance the innate adhesion properties of the material by enabling suction forces. The adhesiveness of the removably-adhesive layer 210 can be restored by washing the removably-adhesive layer 210 to remove dirt from the layer and to clear the pores of a suction-cup micropattern that has been etched, cast, or printed onto the skin-facing surface of the removably-adhesive layer 210.

With continued reference to FIG. 6A, the removably-adhesive layer 210 can be characterized by a thickness dimension 254 that corresponds to the span between the skin-facing surface 212 and the device-facing surface 211 of the adhesive layer 210. In some arrangements, the thickness dimension 254 can be a value between 1 mm and 10 mm. Each void 250 can be characterized by a void-depth dimension 256 that corresponds to the distance the void 250 extends into the adhesive layer 210 from the skin-facing surface 212. In some arrangements, the void-depth dimension 256 can be a value between 50 μm and 1000 μm. In some aspects, the removably-adhesive layer 210 can be characterized by a void-volume ratio that is defined as the ratio of the volume of the voids 250 compared to the volume of the remaining adhesive layer 210. In some arrangements, the removably-adhesive layer 210 can have a void-volume ratio having a value of: 0.05, 0.1, 0.2, 0.25, 0.3, 0.4 or a value between any of these values.

FIG. 6B illustrates a schematic representation of an enlarged top view of the skin-facing surface 212 of the removably-adhesive layer 210. As shown in FIG. 6B, the adhesive layer 210 can include a plurality of circular-shaped openings that are formed by the voids 250. In some aspects, the size and distribution of the voids 250 can be tailored to achieve a secure, yet easily movable, attachment between the wearable device 200 and the skin of the user. In some aspects, the adhesive layer 210 can be characterized by a void-area ratio defined as the ratio between the surface area of the voids 250 compared to the surface area of the remaining adhesive layer 210. For example, FIG. 6B shows a removably-adhesive layer 210 with a void-area ratio of about 0.2 because the area of the white circles is about one-fifth the area of the remaining darkened rectangle. In some aspects, the removably-adhesive layer 210 can have a void-area ratio having a value of: 0.01, 0.05, 0.1, 0.15, 0.2, 0.3, 0.4 or a value between any of these values. In some aspects, the adhesive layer 210 can be characterized by the range and frequency of the void diameters 252.

FIG. 6C depicts a side cross-sectional view of an illustrative, non-limiting example of a removably-adhesive layer 210, according to some aspects of the present disclosure. As shown in FIG. 6C the removably-adhesive layer 210 can include voids 250 that define a sharp-edged well structure rather than, or in addition to, the curvilinear-edged well structures indicated in FIG. 6A. As described with regard to FIG. 6A, the voids 250 can have a void depth dimension 256 that corresponds the extent to which the void 250 extends into the removably-adhesive layer 210 from the skin-facing surface 212 of the removably-adhesive layer 210.

FIGS. 6D and 6E illustrate schematic representations of an enlarged top view of the skin-facing surface 212 of the removably-adhesive layer 210 to show non-limiting, illustrative examples of arrangements of voids 250 that can be used to achieve the suction-cup-like adhesive properties of the removably-adhesive layer 210. FIG. 6D illustrates that the voids 250 can be arranged as in ordered array of cylindrical (or hemi-spherical) wells. The dashed lines in FIG. 6D indicate that the centers of the circular openings of the voids 250 need not be aligned with the centers of the voids 250 in an adjacent row of the array of voids 250. FIG. 6E illustrates that the voids 250 can take on geometric shapes other than a circle, such as, for example the “T-shaped” hammerhead structure shown in FIG. 6E.

As can be appreciated from viewing FIG. 6A or 6C inverted, the removably-adhesive layer 210 can be patterned by molding the removably-adhesive layer 210 over a surface having protrusions in the shape that is the inverse of the voids 250. For example, a mold can be printed (e.g., three-dimensional (3D) printed), etched, or otherwise constructed to have a pattern (or nanopattern) that is the inverse of the desired arrangements of the voids 250. The mold can be produced using a 3D printer capable of producing equally spaced octopus-like suction cups in an array of equally spaced (e.g., 50 μm, 200 μm, 1000 μm) suction cups (e.g., voids 250). In some arrangements, the mold can possess a hammerhead shaped indent of the same diameter, again to allow suction forces upon the skin. As described herein, the shape, size, and design of the voids 250 can be designed according to the desired adhesiveness of the removably-adhesive layer 210. The mold can be made from silicon or other suitable 3D printing material. The curing of the removably-adhesive layer 210 can be initiated by ultraviolet light when the uncured layer material is in the mold.

As described herein, the removably-adhesive layer 210 can be made initially as an uncured, flowable material that can be placed into a mold. A non-limiting, illustrative example of a scalable method of making a suitable material is presented as follows. Two grams of acrylamide can be dissolved in a minimum amount of distilled water (e.g., 1-2 ml) along with 0.4 grams of acrylic acid, sixteen milligrams of MBAA (N,N′ Methylenebis(acrylamide) and 0.334 ml of 2-Hydroxy-2-methylpropiophenone). This solution can be brought up to a volume of ten milliliters using glycerol. The solution can then be purged of oxygen by bubbling nitrogen through the solution for ten minutes. To prevent the material from being bound to the surface the solution is placed on during synthesis, the holding vessel can be coated with an inert substance (e.g., PTFE). The solution can then be poured over the desired mold and subjected to a UV light spectrum (e.g., 365 nm) for 10-30 minutes for a layer having a thickness of 2-3 mm. The material can be washed to remove unreacted reagents and facilitate in removal and handling of the material.

### Software

In some aspects, the system 100 (e.g., the processor unit 205) of the present disclosure can use a custom signal-processing and machine-learning pipeline to quantify muscle fatigue during an aerobic or anaerobic exercise, as described herein. Continuous monitoring of local muscle fatigue during performance of work is possible by measuring myoelectric activity of particular muscles by sEMG. The system 100 can detect muscle fatigue from analysis of sEMG signals received from the wearable device 200 during anaerobic or aerobic exercise.

FIG. 7A illustrates the wearable device 200 in use to monitor an sEMG signal of an underlying muscle 6 through the skin 2 to which the wearable device 200 is secured by the removably-adhesive layer 210. In some aspects, the wearable device 200 can be configured (e.g., can include a processor unit 205 that has a processor configured) to pre-process the sEMG signal before providing data to the algorithm to ensure that the signal is clean and free of unwanted artifacts. The system 100 (e.g,, the processor unit 205) can apply a high-pass filter 522 (e.g., with a cutoff frequency of 10 Hz) to the sEMG signal 502 to remove low-frequency components (e.g., to remove motion artifacts). The system 100 can apply subsequently a low-pass filter 524 (e.g., with a cutoff frequency of 800 Hz) to remove high-frequency components (e.g., to improve signal quality). After filtering, the clean signal can be further processed with Short Time Fourier Transform (STFT) to obtain a time-frequency representation of the EMG signal, as described herein. In some arrangements, a windowing step can be applied in which the sEMG signal 502 is divided into a plurality of time windows 526, thereby facilitating signal analysis in limited time intervals. In some aspects, the windowing step can be essential for extracting information regarding muscle fatigue at a precise moment. Each time window 526 can represent a “snapshot” of the muscle activity, providing a picture of muscle conditions at a specific time. In some arrangements, the system 100 can be configured for real-time monitoring of muscle fatigue by extracting a signal from a time window 526 and processing directly the extracted signal (e.g., by STFT) directly with the device 200, as described herein, to extract muscle activity information in a specific time frame.

FIG. 7B illustrates a schematic representation of a signal-processing method of the system 100, according to some aspects of the present disclosure. The signal-processing method can be performed locally by the wearable device 200 using a processor of the processor unit 205. The signal-processing method can include segmenting the filtered signal 503 into one or more spans of time (e.g., a time window 526). The system 100 can analyze the signal within that span of time, for example, by performing a Short-Time Fourier Transform on the extracted portion of the signal, as indicated by the transform module 528 depicted in FIG. 7B. From the STFT obtained for the time window 526, a spectrogram 530 of the STFT can be input into a neural network to obtain muscle fatigue estimates, as described herein. In some arrangements, both the magnitude and the phase information of the STFT output can be used in complex valued layers of neural networks to obtain muscle fatigue estimates.

In some arrangements, the system 100 can use a neural network (e.g. a convolutional neural network) to assess fatigue levels of muscles during exercise. In some aspects, the system 100 can include a neural network (e.g., encoded within the processor unit 205) that has convolutional layers to process the two-dimensional STFT input from the device 200. In some aspects, the system 100 can track the fatigue states of specific muscles during workouts and compare those levels to a user's baseline to check whether these levels deviate significantly from the user's typical fatigue patterns for the muscle examined and warn the user about risk of injury. In some arrangements, the system 100 can determine whether the user is undertraining or overtraining for the user's specific fitness goals. The system 100 can allow for personalized, real-time adjustments to the user's workout, such as, for example, stopping an exercise when the muscle reaches an optimal level of exertion for the user's training goal. The system 100 can generate, or allow a user to generate, customized workout planning. In some arrangements, the system 100 can provide the user with recommendations for specific exercises (e.g., the number of sets to perform in future sessions, the optimal timing for the next session to ensure proper muscle recovery). The neural network of the system 100 can be fine-tuned on user-specific sEMG data collected during these training sessions, improving the accuracy of the muscle-fatigue-estimation algorithm.

FIG. 7C illustrates a method by which the system 100 can use a neural network to assess muscle fatigue of a user of the wearable device 200 based on a sEMG signal detected by the device 200 during a muscle exertion (e.g., workout) of the user. In some arrangements, the assessment can be performed locally on the wearable device 200 (e.g., using the processor unit 205). In some aspects, the system 100 can include a neural network (e.g., a convolutional neural network 535) that receives input information (e.g., a spectrogram 530) derived from a sEMG signal detected with the wearable device 200. The neural network can be run on a chip within the wearable device 200 or can be accessible through the remote server 600. The system 100 can be configured to return a muscle-fatigue indication 560, such as by displaying a gage or a numerical value between 0 and 100 to indicate a percentage of muscle fatigue of the muscle being monitored by the system 100. In some aspects, the system 100 can be configured to account for the temporal evolution of muscle fatigue. For example, the neural network of the system 100 can incorporate Recurrent Neural Network (RNN) layers, such as, for example, Long Short-Term Memory (LSTM) or Gated Recurrent Units (GRUs). These RNN layers can be configured to process the data output from the CNN layers, maintaining a memory of previous inputs and allowing the network to understand the temporal progression of muscle fatigue. In some aspects, by capturing the temporal dependencies in the spectrogram features, the RNN layers can help identify patterns characteristic of muscle fatigue over time.

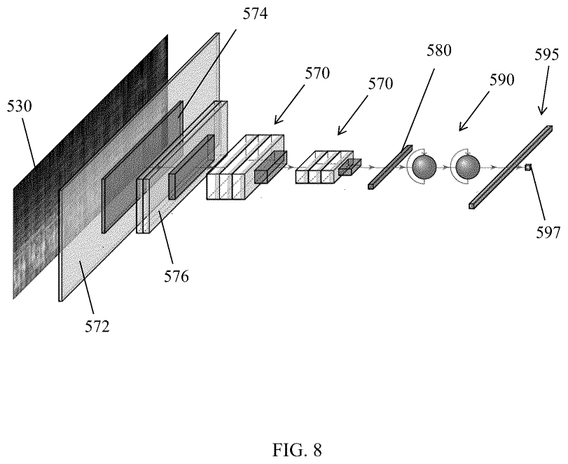

FIG. 8 depicts an illustrative, non-limiting method by which the system 100 can use a neural network analysis to estimate muscle fatigue from sEMG data. The spectrogram 530 of the sEMG signal at a given moment (e.g., obtained from STFT, as described herein) can be used as input to a convolution neural network. The network begins with one or more initial convolutional layers 572, which are followed by an activation function such as ReLU, sigmoid, or hyperbolic tangent (tanh) or others, followed by a max pooling layer 574, as shown in FIG. 8. The convolutional layers may also include a batch normalization layer, depending on the specific implementation. This sequence 570 of convolution, activation, batch normalization, and pooling can be repeated iteratively, reducing the array dimensions in the X and Y planes while expanding the depth (Z dimension) to capture hierarchical features. After the convolutional blocks, the output can be flattened to convert the feature maps into a one-dimensional (1D) feature vector 580, which can then be fed into one or more recurrent neural network (RNN) layers 590. These RNN layers 590 can include Long Short-Term Memory (LSTM) units, Gated Recurrent Units (GRU), or other variants designed to capture temporal dependencies in the data. Following the RNN layers, one or more fully-connected linear layers 595 are used to aggregate the features and generate the final muscle fatigue estimate 597. Additionally, dropout layers can be included throughout the architecture, particularly after the fully-connected layers, to prevent overfitting by randomly deactivating a fraction of neurons during training. This neural network architecture allows the system 100 to extract both spatial and temporal features from the sEMG data for accurate muscle fatigue estimation.

### Other Variations and Terminology

While certain embodiments have been described, these embodiments have been presented by way of example only and are not intended to limit the scope of protection. Indeed, the novel methods and systems described herein may be embodied in a variety of other forms. It will be understood by those skilled in the art that the present disclosure extends beyond the specifically disclosed embodiments to other alternative embodiments or uses and obvious modifications and equivalents thereof, including embodiments which do not provide all of the features and advantages set forth herein. Furthermore, various omissions, substitutions, and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed; others may be added. Accordingly, the scope of the present disclosure is not intended to be limited by the specific disclosures of preferred embodiments herein and may be defined by claims as presented herein or as presented in the future. The language of the claims is to be interpreted broadly based on the language employed in the claims and not limited to the examples described in the patent specification of during prosecution of the application, which examples are to be construed as non-exclusive.

Features, materials, characteristics, or groups described in conjunction with a particular aspect, embodiment, or example are to be understood to be applicable to any other aspect, embodiment, or example described herein unless incompatible therewith. All of the features disclosed in this specification (including any accompanying claims, abstract, and drawings), or all of the steps of any method or process so disclosed, may be combined in any combination, except combinations where at least some of such features or steps are mutually exclusive. The protection extends to any novel one, or any novel combination, of the features disclosed in this specification (including any accompanying claims, abstract, and drawings), or to any novel one, or any novel combination, of the steps of any method or process so disclosed.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of the stated amount. As another example, the terms “generally parallel” and “substantially parallel” may refer to a value, amount, or characteristic that departs from exactly parallel by less than 15 degrees.

What is claimed is:

1. A device for accessing a muscle-fatigue status of a muscle of a user, the device comprising:
a removably-adhesive layer disposed on an outer surface of the device, a user-facing surface of the removably-adhesive layer configured to removably adhere the device to a skin of the user;
a pair of bi-polar electrodes disposed on the outer surface of the device and protruding through the user-facing surface of the removably-adhesive layer, the pair of bi-polar electrodes configured to detect a surface electromyography (sEMG) signal of the muscle of the user; and
a processing unit configured to analyze the sEMG signal and further configured to determine the muscle-fatigue status of the muscle of the user.

2. The device of claim 1, wherein the processing unit is configured to analyze a window of time of the sEMG signal by performing a short-time Fourier transform of the sEMG signal for the window of time.

3. The device of claim 1, wherein the processing unit is configured to determine a spectrogram of the sEMG signal.

4. The device of claim 3, wherein the processing unit comprises a neural network configured to analyze the spectrogram of the sEMG signal.

5. The device of claim 1, wherein the adhesive layer comprises a hydrogel network.

6. The device of claim 1, wherein the device comprises a flexible printed circuit board and a flexible housing.

7. The device of claim 1, wherein the user-facing surface comprises a plurality of semi-spherical indentations.

8. The device of claim 7, wherein a depth dimension of the semi-spherical indentations is within a range of 50 microns to 1000 microns.

9. A system for determining a muscle-fatigue estimate for a muscle of a user, the system comprising:
a wearable device configured to adhere to a skin of the user and further configured to detect a surface electromyography (sEMG) signal of the muscle;
a processor configured to generate a spectrogram from the sEMG signal;
a neural network configured to receive the spectrogram as an input and to return as an output a value of the muscle-fatigue estimate.

10. The system of claim 9, wherein the neural network is encoded on a microchip disposed within the wearable device.

11. The system of claim 9, wherein the wearable device comprises a flexible printed circuit board.

12. The system of claim 9, wherein the neural network comprises one or more recurrent neural network layers.

13. The system of claim 9, further comprising a remote server configured to receive data from the wearable device, the remote server further configured to compile a fatigue history for the user from the data.

14. The system of claim 13, further comprising an app configured to display on a graphical user interface a personalized real-time recommendation.

15. The system of claim 14, wherein the personalized real-time recommendation is selected from the group consisting of: an injury warning, an undertraining determination, a cessation of an exercise, a customized workout, a recommended exercise, a recommended number of sets to perform in future sessions, an optimal timing for a next training session.

16. The system of claim 15, wherein the personalized real-time recommendation is updated based on the fatigue history of the user.

17. A method for accessing a muscle-fatigue status of a muscle of a person, the method comprising:
adhering a wearable device to a skin of the person;
detecting with the wearable device a surface electromyography (sEMG) signal of the muscle;
generating a spectrogram from the sEMG signal;
inputting the spectrogram into a neural network configured to determine the muscle-fatigue status of the muscle based on the spectrogram; and
displaying on a graphical user interface an indication of the muscle-fatigue status.

18. The method of claim 17, wherein the neural network is run on a chip within the wearable device.

19. The method of claim 17, wherein the method further comprises comparing the muscle-fatigue status to a fatigue history compiled for the user over a series of workouts.

20. The method of claim 19, wherein the fatigue history is stored on a remote server.
